(19) United States
(12) Patent Application Publication
(10) Pub. No.: US 2021/0393246 A1
(43) Pub. Date: Dec. 23, 2021

(54) ELONGATED MEDICAL ASSEMBLY HAVING SELECTIVELY EXPANDABLE-AND-CONTRACTIBLE ASSEMBLY

(71) Applicant: Baylis Medical Company Inc., Montreal (CA)

(72) Inventors: Charlene Leung, Scarborough (CA); Gareth Davies, Toronto (CA)

(21) Appl. No.: 17/340,615

(22) Filed: Jun. 7, 2021

Related U.S. Application Data
(60) Provisional application No. 63/040,577, filed on Jun. 18, 2020.

(51) Int. Cl. A61B 17/00

(52) U.S. Cl. CPC A61B 17/00234; A61B 17/0057; A61B 2017/00247; A61B 2017/00672; A61B 2017/00592

(57) ABSTRACT

An elongated medical assembly has a distal region configured to be positioned, at least in part, proximate to a biological feature of a patient. A puncture assembly is configured to be located proximate to the distal region. A selectively expandable-and-contractible assembly is located proximate to the distal region.

### TECHNICAL FIELD

This document relates to the technical field of (and is not limited to) a synergistic combination of an elongated medical assembly, a puncture assembly and a selectively expandable-and-contractible assembly (and method therefor).

### BACKGROUND

Known medical devices are configured to facilitate a medical procedure, and help healthcare providers diagnose and/or treat medical conditions of sick patients.

### SUMMARY

It will be appreciated that there exists a need to mitigate (at least in part) at least one problem associated with the existing (known) elongated medical assemblies (also called the existing technology). After much study of, and experimentation with, the existing (known) elongated medical assemblies, an understanding (at least in part) of the problem and its solution have been identified (at least in part) and are articulated (at least in part) as follows:

Treatment of cardiac disease and/or ailments in the left atrium (LA) of the heart may require access from the right atrium (RA) of the heart by means of a puncture (such as a transseptal puncture) through a biological wall or biological feature (such as the fossa ovalis of the heart) to enable the delivery of a therapeutic device. In certain procedures, the position of the puncture site (on the fossa ovalis) may be critical for the accurate delivery of the therapeutic device to a desired anatomy (biological feature). Accurate placement of the puncture device (such as, a needle or a radio frequency wire, etc.) may be dependent on the ability to extend the reach of (and/or the positioning of) the puncture device. The puncture device may be delivered by a sheath, a dilator or a combination thereof, etc.). Procedural issues and/or inefficiencies may arise when the tip of the medical device (such as a sheath assembly) cannot reach or be positioned at the desired site. Tenting of the septum (formation of a tent in the septum) is a method used to visualize a potential puncture site for the puncture device on the fossa ovalis (a biological feature, etc.). However, there may exist concerns that this method may result in an accidental puncture. Unfortunately, prolapsing of (collapsing of) the medical device (the sheath and dilator combination) may arise during the formation of the puncture.

It may be desirable to provide a medical device (amongst other types or arrangements) having a distal tip that may be positioned with sufficient precision and/or reach, stabilized during formation of the puncture hole, and/or accurately, intuitively controlled by the surgeon.

It may be desirable to provide an arrangement (amongst other types or arrangements) for directing, stabilizing and/or visualizing a medical device during a procedure.

It may be desirable to provide an arrangement (amongst other types or arrangements) having an inflatable balloon configured to treat coronary and peripheral vasculature disease where the balloon is inflated to enlarge or dilate the artery area with stenosis. The inflatable balloon may be used to dilate the septum, etc.

To mitigate, at least in part, at least one problem associated with the existing technology, there is provided (in accordance with a major aspect) an apparatus. The apparatus includes and is not limited to (comprises) an elongated medical assembly having a distal region (tip or distal portion, etc). A puncture assembly is located proximate to the distal region. A selectively expandable-and-contractible assembly is located proximate to the distal region.

To mitigate, at least in part, at least one problem associated with the existing technology, there is provided (in accordance with a major aspect) an apparatus. The apparatus includes and is not limited to (comprises) an elongated medical assembly having a distal region configured to be positioned, at least in part, proximate to a biological feature of a patient. The elongated medical assembly has a proximal region spaced apart from the distal region. The proximal region is configured to be positioned outside of the patient (after the distal region, in use, is positioned, at least in part, in the biological feature of the patient). A puncture assembly is configured to be located proximate to the distal region. The puncture assembly is configured to form a puncture hole through the biological feature (after the distal region, in use, is positioned, at least in part, proximate to the biological feature). A selectively expandable-and-contractible assembly is configured to be deployable at (from) the distal region in (from) a contracted condition to an expanded condition. In the contracted condition, the selectively expandable-and-contractible assembly is contained within the distal region. In the expanded condition, the selectively expandable-and-contractible assembly is deployed, at least in part, from the distal region and proximate to the biological feature.

To mitigate, at least in part, at least one problem associated with the existing technology, there is provided (in accordance with a major aspect) a method. The method is for using an elongated medical assembly having a distal region configured to be positioned, at least in part, proximate to a biological feature of a patient, and a puncture assembly located proximate to the distal region, and a selectively expandable-and-contractible assembly located proximate to the distal region. The method includes and is not limited to (comprises) deploying the selectively expandable-and-contractible assembly from the distal region in (from) a storage condition (a standby condition, an uninflated condition, etc.) to a deployment condition, in which the selectively expandable-and-contractible assembly is deployed, at least in part, from the distal region to a position located proximate to the biological feature. In the storage condition, the selectively expandable-and-contractible assembly is contained within the distal region. In the deployment condition, the selectively expandable-and-contractible assembly is deployed, at least in part, from (removed from) the distal region and to a position located proximate to the biological feature.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1, FIG. 2 and FIG. 3 depict side views of embodiments of an elongated medical assembly, a puncture assembly and a selectively expandable-and-contractible assembly; and

FIG. 4, FIG. 5 and FIG. 6 depict side views of embodiments of the elongated medical assembly, the puncture assembly and the selectively expandable-and-contractible assembly of FIG. 1; and

FIG. 7, FIG. 8 and FIG. 9 depict side views of embodiments of the elongated medical assembly, the puncture assembly and the selectively expandable-and-contractible assembly of FIG. 1; and

FIG. 10 and FIG. 11 depict perspective side views of embodiments of the elongated medical assembly, the puncture assembly and the selectively expandable-and-contractible assembly of FIG. 1; and

FIG. 12 and FIG. 13 depict side views of embodiments of the selectively expandable-and-contractible assembly of FIG. 1.

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

LISTING OF REFERENCE NUMERALS
USED IN THE DRAWINGS

| | |
|---|---|
| medical assembly | 102 |
| distal region | 104 |
| proximal region | 105 |
| puncture assembly | 106 |
| puncture hole | 107 |
| contractible assembly | 108 |
| deflecting member | 109 |
| sac assembly | 110 |
| sac assemblies | (110A, 110B) |
| stabilization member | 111 |
| inflation lumen | 112 |
| elongated guidewire | 200 |
| mesh structure | 502 |
| inflation device | 700 |
| sheath assembly | 400 |
| guidewire lumen | 401 |
| handle | 402 |
| mechanically-expandable | 500 |
| member | |
| action force | 800 |
| reaction force | 802 |
| biological feature | 900 |
| patient | 902 |
| septum | 904 |
| right atrium | 906 |

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the disclosure is defined by the claims. For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the disclosure is limited to the subject matter provided by the claims, and that the disclosure is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

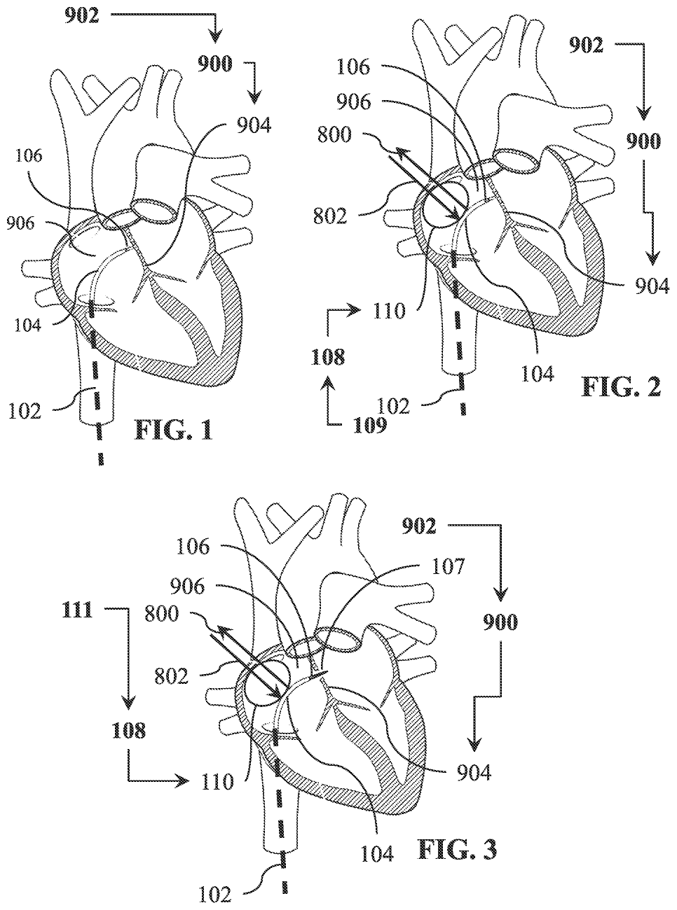

FIG. 1, FIG. 2 and FIG. 3 depict side views of embodiments of an elongated medical assembly 102, a puncture assembly 106 and a selectively expandable-and-contractible assembly 108. It will be appreciated that FIG. 1, FIG. 2 and FIG. 3 depict cross-sectional views of a biological feature 900 with the devices superimposed thereon.

Referring to the embodiments as depicted in FIG. 1, FIG. 2 and FIG. 3, there is depicted an embodiment of an elongated medical assembly 102 having a distal region 104. The distal region 104 is configured to be positioned, at least in part, proximate to a biological feature 900 of a patient 902. The biological feature 900 may include a septum 904 (a biological wall) of the heart of the patient 902, etc. A puncture assembly 106 is configured to be located (preferably, mounted) proximate to the distal region 104 of the elongated medical assembly 102. The puncture assembly 106 is configured to form a puncture hole 107 (as depicted in FIG. 3) through the biological feature 900. This is done (preferably) after the distal region 104, in use, is positioned, at least in part, proximate to the biological feature 900. The puncture hole 107 includes (preferably) a transseptal puncture. A selectively expandable-and-contractible assembly 108 is configured to be mounted to, and deployable from, the distal region 104. Preferably, the selectively expandable-and-contractible assembly 108 and the puncture assembly 106 are positioned proximate to each other.

Referring to the embodiments as depicted in FIG. 1, FIG. 2 and FIG. 3, the selectively expandable-and-contractible assembly 108 includes (preferably) a sac assembly 110, an inflatable and deflatable balloon, and/or any equivalent thereof. It will be appreciated that the selectively expandable-and-contractible assembly 108 may include any type of mechanically expandable frame, device, etc. The sac assembly 110 is configured to be deployable from (at) the distal region 104 in (from) an uninflated condition (or a non-deployed condition, as depicted in FIG. 1) to an inflated condition (a deployed condition, as depicted in FIG. 2 and FIG. 3).

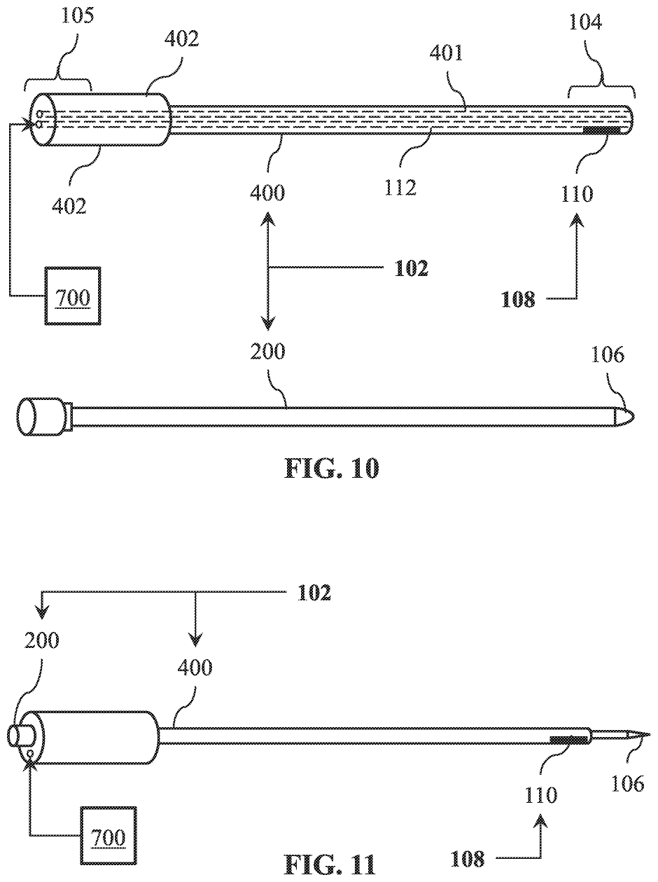

Referring to the embodiments as depicted in FIG. 1, FIG. 2 and FIG. 3, the sac assembly 110 is embedded in (or mounted to) the distal region 104 of the elongated medical assembly 102. Specific embodiments of the elongated medical assembly 102 are depicted in FIG. 10 and FIG. 11. The sac assembly 110 may be embedded on one side of a (or around the entire) circumference of the elongated medical assembly 102, etc. Once the elongated medical assembly 102 has been positioned in the right atrium 906, the sac assembly 110 may be expanded (actuated) by the injection of fluid through an inflation lumen 112 (as depicted in FIG. 10). The inflation lumen 112 may be embedded in (and along) a wall of the elongated medical assembly 102. The inflation lumen 112 may be fluidly connected to an inflation device 700 (as depicted in FIG. 10), such as a syringe, an insufflator or a pump positioned outside the body of the patient 902. The action force 800 applied by the expanded sac assembly 110 against the atrium wall surrounding the right atrium 906 creates, at least in part, a reaction force 802 on the tip (distal portion or distal section) of the elongated medical assembly 102 that can cause it to deflect, thereby changing the reach and/or orientation (of the elongated medical assembly 102). The expanded sac assembly 110 resting against the atrium wall surrounding the right atrium 906 can also stabilize the elongated medical assembly 102 during transseptal puncture to prevent prolapsing. This stabilization better maintains, at least in part, the position of the tip (distal portion or distal section) of the elongated medical assembly 102 on the septum and improves the translation of forces applied to the puncture device against the septum thereby providing a more predictable puncture. Furthermore, the expandable sac assembly 110 may be visible under fluoroscopy when filled with a contrast fluid, etc. The selectively expandable-and-contractible assembly 108 is configured to deflect, stabilize and/or visualize, at least in part, a portion or section of the elongated medical assembly 102 while the elongated medical assembly 102 is activated or used to form a puncture hole 107 (such as a transseptal puncture).

Referring to the embodiments as depicted in FIG. 1, FIG. 2 and FIG. 3, the selectively expandable-and-contractible assembly 108 (or the expandable sac assembly 110) is configured to deflect, at least in part, the distal region 104 of the elongated medical assembly 102. The expandable-and-contractible assembly 108 (or the expandable sac assembly 110) is mounted to (preferably, embedded on, or along) a side of the elongated medical assembly 102. This is done (preferably) such that the expandable-and-contractible assembly 108 does not surround an entire circumference of the elongated medical assembly 102. That is, the expandable-and-contractible assembly 108 is mounted to a portion of the elongated medical assembly 102 (such as, along, at least in part, half of the circumference of the elongated medical assembly 102). The elongated medical assembly 102 may be straight or shaped with a curve, etc. The expandable sac assembly 110 is (preferably) embedded on the opposite side of the direction of the curve of the elongated medical assembly 102. The center of the expandable sac assembly 110 may be embedded about two (2) to about eight (8) centimeters from the tip (distal portion or distal section) of the elongated medical assembly 102. The expandable sac assembly 110 is connected to an inflation lumen 112 (as depicted in FIG. 10). It will be appreciated that the inflation lumen 112 (depicted in FIG. 10) may exit the sheath wall (of the elongated sheath assembly 400 at a distal portion thereof) into the expandable sac assembly 110 (if desired). In accordance with an option (not depicted) the expandable sac assembly 110 is configured to slide (move) over an elongated introducer sheath, etc. The inflation lumen 112 is (preferably) embedded (positioned) behind or adjacent to the outer wall (outer surface) of the elongated medical assembly 102. The inflation lumen 112 exits the elongated medical assembly 102 at the proximal region 105 (as depicted in FIG. 10). With the elongated medical assembly 102 positioned in the right atrium 906, a fluid may be injected through the inflation lumen 112 (as depicted in FIG. 10) to fill the expandable sac assembly 110 until the expandable sac assembly 110 touches the atrium wall surrounding the right atrium 906 (as depicted in FIG. 2). Additional fluid may be injected to increase the interior volume of the sac assembly 110. This is done (preferably) such that the action force 800 of the expanded sac assembly 110 may be applied against a wall (at least one wall) of the right atrium 906 creating a reaction force 802 that pushes, in use, the distal region 104 (or the puncture assembly 106) downward and towards the septum 904 (a biological wall). Once positioned on the septum 904, the puncture assembly 106 may be utilized for performing the transseptal puncture through the fossa ovalis. It will be appreciated that the action force 800 may include a distribution of forces (such as, a partially circumferential distribution of forces) applied by a surface feature (such as, a round surface area) of the selectively expandable-and-contractible assembly 108 (such as, a balloon, etc.) against the tissue or biological feature. It will be appreciated that the action force 800 may include a single point force, etc.

Referring to the embodiment as depicted in FIG. 2, the sac assembly 110 is also configured to impart (apply) an action force 800 against the biological feature 900 (such as the right atrium wall surrounding the right atrium 906) after the sac assembly 110 is deployed. The action force 800, in use, urges a reaction force 802 to become imparted to the distal region 104, which causes the distal region 104 to deflect, thereby changing the reach of the distal region 104.

Referring to the embodiment as depicted in FIG. 2, the sac assembly 110 is also configured to rest against the biological feature 900 (such as, the right atrium wall surrounding the right atrium 906) to stabilize the elongated medical assembly 102 during deployment of the puncture assembly 106 (such as for puncturing of the septum 904, thereby prevent, at least in part, prolapsing of the right atrium 906 of the heart).

Referring to the embodiment as depicted in FIG. 1, the biological feature 900 includes (and is not limited to) the right atrium 906 of the heart, etc.

Referring to the embodiments as depicted in FIG. 1, FIG. 2 and FIG. 3, there is depicted a method of using the elongated medical assembly 102, the puncture assembly 106 and the selectively expandable-and-contractible assembly 108. The method includes deploying the selectively expandable-and-contractible assembly 108, at (from) the distal region 104, from a storage condition to a deployment condition. In the storage condition, the selectively expandable-and-contractible assembly 108 is contained within (stored within) the distal region 104. In the deployment condition, the selectively expandable-and-contractible assembly 108 is deployed, at least in part, from the distal region 104 to a position located proximate to the biological feature 900.

Referring to the embodiment as depicted in FIG. 2, in accordance with an embodiment, an apparatus includes and is not limited to (comprises) an elongated medical assembly 102 having a distal region 104. A puncture assembly 106 is located proximate to the distal region 104. A deflecting member 109 is located proximate to the distal region 104. The deflecting member 109 is configured to selectively adjust, at least in part, a position of the distal region 104 of the elongated medical assembly 102. It will be appreciated that the selectively expandable-and-contractible assembly 108 is configured to selectively adjust, at least in part, a position of the distal region 104 of the elongated medical assembly 102 (this is done, preferably, after the elongated medical assembly 102 is positioned proximate to the biological feature 900, and the expandable-and-contractible assembly 108 is deployed or activated accordingly).

Referring to the embodiment as depicted in FIG. 3, in accordance with an embodiment, an apparatus includes and is not limited to (comprises) an elongated medical assembly 102 having a distal region 104. A puncture assembly 106 is located proximate to the distal region 104. A stabilization member 111 is located proximate to the distal region 104. The stabilization member 111 is configured to stabilize a position of the distal region 104 of the elongated medical assembly 102. It will be appreciated that the selectively expandable-and-contractible assembly 108 is configured to selectively stabilize, at least in part, a position of the distal region 104 of the elongated medical assembly 102 (this is done, preferably, after the elongated medical assembly 102 is positioned proximate to the biological feature 900, and the expandable-and-contractible assembly 108 is deployed or activated accordingly).

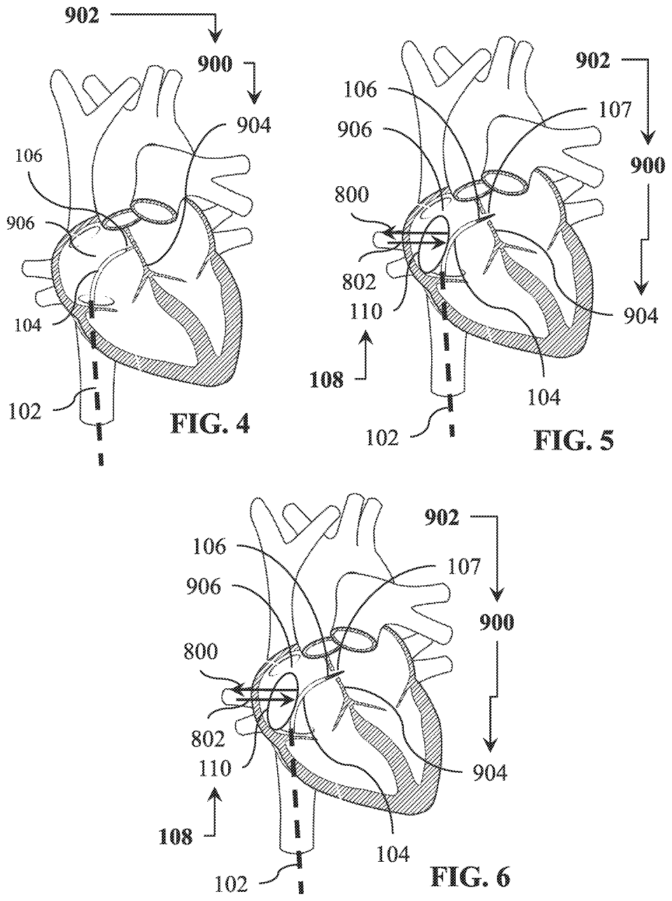

FIG. 4, FIG. 5 and FIG. 6 depict side views of embodiments of the elongated medical assembly 102, the puncture assembly 106 and the selectively expandable-and-contractible assembly 108 of FIG. 1. It will be appreciated that FIG. 4, FIG. 5 and FIG. 6 depict cross-sectional views of a biological feature 900 with the devices superimposed thereon

Referring to the embodiments as depicted in FIG. 4, FIG. 5 and FIG. 6, the selectively expandable-and-contractible assembly 108 (or the expandable sac assembly 110) is configured to extend (reach) and/or provide additional stability of the elongated medical assembly 102. The selectively expandable-and-contractible assembly 108 (or the expandable sac assembly 110) is mounted to (preferably, embedded to) a side section of the elongated medical assembly 102. This is done (preferably) such that the selectively expandable-and-contractible assembly 108 (or the expandable sac assembly 110) does not surround an entire circumference of the elongated medical assembly 102. The selectively expandable-and-contractible assembly 108 (or the expandable sac assembly 110) surrounds a portion of the elongated medical assembly 102. The selectively expandable-and-contractible assembly 108 (or the expandable sac assembly 110) surrounds (preferably) about half of the circumference of the elongated medical assembly 102. The expandable sac assembly 110 may be located on a side section of the elongated medical assembly 102, the furthest away from the septum 904 (once the elongated medical assembly 102 is positioned proximate to the septum 904). This condition or position may be verified under fluoroscopy by injecting a contrast agent into the expandable sac assembly 110, etc. The position of the expandable sac assembly 110 along the elongated medical assembly 102 is such that the expandable sac assembly 110 sits in the inferior vena cava (IVC) or at the orifice where the IVC enters into the right atrium 906. The center of the expandable sac assembly 110 may be embedded about three (3) to about ten (10) centimeters (cm) from the distal region 104 of the elongated medical assembly 102. The expandable sac assembly 110 is configured to be connected to the inflation lumen 112 (as depicted in FIG. 10). The inflation lumen 112 is embedded in an outer wall of the elongated medical assembly 102. The inflation lumen 112 exits (is aligned along an elongated length of) the elongated medical assembly 102. After the elongated medical assembly 102 is positioned proximate to the septum 904 (and is positioned on the fossa ovalis), a fluid may be injected through the inflation lumen 112 (as depicted in FIG. 10) to fill the expandable sac assembly 110 until the expandable sac assembly 110 touches the wall of the inferior vena cava (IVC) and/or the right atrium 906 of the heart. Additional fluid may be injected into the expandable sac assembly 110 such that the action force 800 of the expandable sac assembly 110 may be applied against the wall or walls of the right atrium 906, and a reaction force 802, if formed or created, may cause the distal region 104 of the elongated medical assembly 102 to reach forward to tent the septum 904. The controlled application of fluid to the expandable sac assembly 110 may tent the septum 904 at a controlled rate and force. Furthermore, the reaction force 802 against the wall surrounding the right atrium 906 may provide stability to the elongated medical assembly 102 during formation of the puncture hole 107 to reduce the risk of prolapse (of the elongated medical assembly 102), etc.

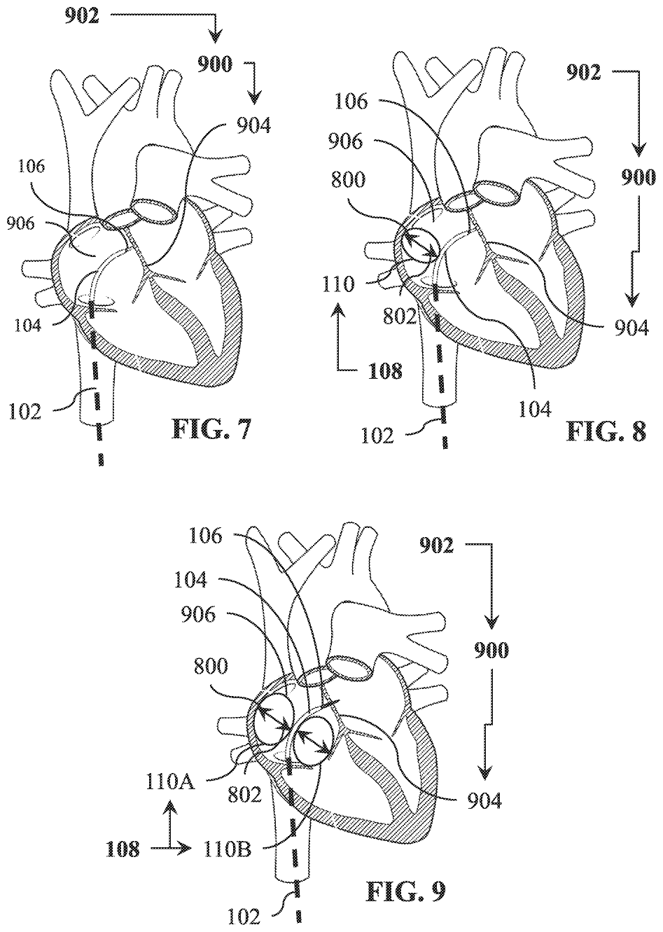

FIG. 7, FIG. 8 and FIG. 9 depict side views of embodiments of the elongated medical assembly 102, the puncture assembly 106 and the selectively expandable-and-contractible assembly 108 of FIG. 1. It will be appreciated that FIG. 7, FIG. 8 and FIG. 9 depict cross-sectional views of a biological feature 900 with the devices superimposed thereon

Referring to the embodiments as depicted in FIG. 7, FIG. 8 and FIG. 9, the selectively expandable-and-contractible assembly 108 (or the expandable sac assembly 110) is configured to act as an anatomical reference tool. Referring to FIG. 9, the sac assembly 110 includes at least one or more expandable sac assemblies (110A, 110B) embedded along a portion or the entire circumference of the elongated medical assembly 102 (as depicted in FIG. 9). The expandable sac assemblies (110A, 110B) may vary in length, diameter and position along the distal region 104 of the elongated medical assembly 102. While the elongated medical assembly 102 is positioned in the right atrium 906, a contrast agent may be injected into one or more of the expandable sac assemblies (110A, 110B) via respective inflation lumens (which may be duplicates of the inflation lumen 112, as depicted in FIG. 10, etc.). The respective inflation lumens may be embedded in (mounted to), at least in part, the elongated medical assembly 102. The expandable sac assemblies (110A, 110B) are configured to touch the inner walls of the right atrium 906. Fluoroscopy may be used to visualize the volume of the deployed expandable sac assemblies (110A, 110B) where the outline of the expandable sac assemblies (110A, 110B) against the walls of the right atrium 906 resemble, to some degree, the outline of the right atrium 906, etc. This condition may help the surgeon infer the position of the elongated medical assembly 102 positioned in the right atrium 906, especially in cases involving an enlarged and/or abnormal anatomy and/or the inability to perform the two-drop technique (known and not described) that may be used to land (position) the elongated medical assembly 102 on the septum 904.

FIG. 10 and FIG. 11 depict perspective side views of embodiments of the elongated medical assembly 102, the puncture assembly 106 and the selectively expandable-and-contractible assembly 108 of FIG. 1.

Referring to the embodiments as depicted in FIG. 10 and FIG. 11, the elongated medical assembly 102 has the distal region 104 (as depicted in FIG. 10 and FIG. 11). The distal region 104 is configured to be positioned, at least in part, proximate to the biological feature 900 of the patient 902 (as depicted in FIG. 1, etc.). The puncture assembly 106 is located proximate to the distal region 104 (as depicted in FIG. 11). A selectively expandable-and-contractible assembly 108 is located proximate to the distal region 104 (as depicted in FIG. 10).

Referring to the embodiments as depicted in FIG. 10 and FIG. 11, the puncture assembly 106 is configured to form the puncture hole 107 (as depicted in FIG. 2, etc.) through the biological feature 900 (such as transseptal puncture, etc.); this is done (preferably) after the distal region 104, in use, is positioned, at least in part, proximate to the biological feature 900 (as depicted in FIG. 2). The selectively expandable-and-contractible assembly 108 is configured to be mounted to, and deployable from, the distal region 104. It is preferred to avoid a pointed tip (distal tip portion) for the puncture assembly 106 (forming or providing a sharp needle). It is preferred to utilize a rounded tip, a blunt tip or a tip configured to reduce, at least in part, atraumatic injury to the tissue, etc.

Referring to the embodiments as depicted in FIG. 10 and FIG. 11, the selectively expandable-and-contractible assembly 108 includes a sac assembly 110. The sac assembly 110 is configured to be deployable from the distal region 104 in (from) an uninflated condition to an inflated condition. The sac assembly 110 may include an inflatable and deflatable balloon, and any equivalent thereof. In the uninflated condition, the sac assembly 110 is contained (positioned, embedded) within the distal region 104. In the inflated condition, the sac assembly 110 is deployed, at least in part, from the distal region 104 and proximate to the biological feature 900.

Referring to the embodiments as depicted in FIG. 10 and FIG. 11, the elongated medical assembly 102 has a proximal region 105 that is spaced apart from the distal region 104. The proximal region 105 is configured to be positioned outside of the patient 902 after the distal region 104, in use, is positioned, at least in part, in the biological feature 900 of the patient 902 (as depicted in FIG. 1, etc.). The selectively expandable-and-contractible assembly 108 is configured to be deployable from the distal region 104 in (from) a contracted condition (as depicted in FIG. 1 and FIG. 10) to an expanded condition (as depicted in FIG. 2). In the contracted condition, the selectively expandable-and-contractible assembly 108 is contained (positioned, embedded) within the distal region 104 (as depicted in FIG. 10). In the expanded condition, the selectively expandable-and-contractible assembly 108 is deployed, at least in part, from the distal region 104 and proximate to the biological feature 900 (as depicted in FIG. 2, etc.). It will be appreciated that the selectively expandable-and-contractible assembly 108 may include many types of arrangements and/or structures, such as (and not limited to) a loop of wire configured to be selectively extended and retracted to perform in an equivalent manner. It is preferred that the selectively expandable-and-contractible assembly 108 is configured to reduce, at least in part, (preferably, to not impact) hemodynamics. Hemodynamics is the dynamics of blood flow. The circulatory system is controlled by homeostatic mechanisms, such as hydraulic circuits are controlled by control systems. Hemodynamic response continuously monitors and adjusts to conditions in the body and its environment.

Referring to the embodiment as depicted in FIG. 10, the elongated medical assembly 102 defines an inflation lumen 112 extending between the distal region 104 and the proximal region 105. The inflation lumen 112 is configured to be fluidly connectable, at the proximal region 105, to the inflation device 700 (which is positioned outside the patient 902, as depicted in FIG. 1).

Referring to the embodiment as depicted in FIG. 10, the sac assembly 110 is also configured to be expanded in response to injection of a fluid movable from the inflation device 700 through the inflation lumen 112 to the sac assembly 110. This is done (preferably) after: (A) the elongated medical assembly 102 is positioned proximate to the biological feature 900; and (B) the inflation lumen 112 is fluidly connected to the inflation device 700; and (C) the inflation device 700 is actuated.

Referring to the embodiment as depicted in FIG. 10, the sac assembly 110 is configured to be embedded on one side of a circumference of the elongated medical assembly 102.

Referring to the embodiment as depicted in FIG. 10, the sac assembly 110 is configured to be embedded around a portion of or the entire circumference of the elongated medical assembly 102.

Referring to the embodiment as depicted in FIG. 10, the inflation lumen 112 is embedded in a wall of the elongated medical assembly 102.

Referring to the embodiments as depicted in FIG. 10 and FIG. 11, the fluid of the inflation device 700 (a fluid source) includes a contrast fluid. The sac assembly 110 is configured to be visible under fluoroscopy when filled with a contrast fluid.

Referring to the embodiments as depicted in FIG. 10 and FIG. 11, the elongated medical assembly 102 may include an elongated sheath assembly 400, a dilator (not shown) and an elongated guidewire 200. The elongated guidewire 200 is configured to be received within the dilator which would be used to insert or guide the sheath assembly 400 into the heart and through which (the dilator) the guidewire would travel. Alternatively, the sheath can be replaced with any device designed to enter the right atrium (RA) for the purposes of performing transseptal puncture

Referring to the embodiment as depicted in FIG. 10, the inflation device 700 includes a syringe, an insufflator or a pump.

Referring to the embodiments as depicted in FIG. 10 and FIG. 11, the guidewire 200 is configured to be inserted into a guidewire lumen 401 provided by the sheath assembly 400. In another embodiment, a dilator (not shown) is inserted into the lumen 401 provided by the sheath assembly 400 and the guidewire 200 is into the dilator. The guidewire lumen 401 is aligned coaxially with the inflation lumen 112. Coaxial means that (A) elements may share the same axis or are rotatable about a common axis (i.e., the elements may be arranged in a concentric manner, the elements may have a coincident axes); or (B) elements may have separate axes that may be aligned parallel to one another. The sheath assembly 400 may include, for instance, polyethylene, polyether block amide (PEBA), a thermoplastic elastomer (TPE), the PEBAX (TRADEMARK) material manufactured by ARKEMA S.A. based in France, the VESTAMID (TRADEMARK) material manufactured by EVONIK INDUSTRIES A. G. based in Germany, etc., and any equivalent thereof. The sheath assembly 400 includes a handle 402.

Referring to the embodiments as depicted in FIG. 10 and FIG. 11, the sac assembly 110 may include polyethylene terephthalate, nylon, polyutherane, silicone, the PEBAX (TRADEMARK) material, etc., and/or any equivalent thereof. The sac assembly 110 may be mounted to the outer wall of the elongated medical assembly 102 (which may include a dilator (not shown) or the sheath assembly 400) by heat bonding using heat shrink, or welding at two ends of the elongated medical assembly 102 (or the sheath assembly 400 or a dilator). This creates an air-tight seal and/or fluid-tight seal between the membrane of the sac assembly 110 and the outer wall of the elongated medical assembly 102, etc. The length and diameter of the sac assembly 110, and the position of the sac assembly 110 along and around the circumference of the elongated medical assembly 102 may dictate the functionality of the sac assembly 110.

Referring to the embodiments as depicted in FIG. 10 and FIG. 11, the sac assembly 110 may be changed to an expandable member, such as a nitinol frame or a flexible mesh structure that is joined to the outer wall of the elongated medical assembly 102 or the sheath assembly 400 or a dilator, and is expanded and/or deployed by a mechanical force, etc. For example, a distal end of the selectively expandable-and-contractible assembly 108 may be fixed to the elongated medical assembly 102 (or the sheath assembly 400) and the proximal end may be attached to a rod (not depicted) that exits the elongated medical assembly 102 or the sheath assembly 400 where a user may push or pull on the rod to expand or retract the expandable member. The expandable member may include a radiopaque element or material, etc.

Referring to the embodiments as depicted in FIG. 10 and FIG. 11, the inflation lumen 112 is embedded into the elongated medical assembly 102. The inflation lumen 112 is configured to be connectable outside of the elongated medical assembly 102 to the inflation device 700. The inflation lumen 112 would exit the sheath wall into the sac assembly 110. The inflation lumen 112 may be embedded in the elongated medical assembly 102 (or the sheath assembly 400 or a dilator) or located on and along the exterior thereof. The inflation lumen 112 is configured to deliver (convey) a fluid (such as air) from the proximal region 105 to the sac assembly 110.

Referring to the embodiments as depicted in FIG. 10 and FIG. 11, for the case where the sac assembly 110 is not utilized, the selectively expandable-and-contractible assembly 108 may include an expandable frame device (or a flexible mesh structure). An elongated flexible actuation rod (member) is inserted into the inflation lumen 112, in which the elongated flexible actuation rod is configured to actuate the expandable frame device, etc.

Referring to the embodiments as depicted in FIG. 10 and FIG. 11, the inflation device 700 may include any type of inflation source, etc. The inflation device 700 may include a syringe, an insufflator and/or a pump mechanism configured to deliver a fluid (such as, a contrast agent and/or saline) to expand the sac assembly 110. The inflation device 700 may include a fluid-filled reservoir located at the proximal region 105 of the elongated medical assembly 102 (which may include a dilator or the sheath assembly 400). The inflation device 700 is configured to provide compressed fluid or to inject a fluid through the inflation lumen 112 to the sac assembly 110.

Referring to the embodiment as depicted in FIG. 10, the elongated medical assembly 102 includes (in accordance with a preferred embodiment) biocompatible material properties suitable for sufficient performance (such as dielectric strength, thermal performance, insulation and corrosion, water and heat resistance), such as for safe performance, for compliance with industrial and regulatory safety standards (or compatible for medical usage), etc. Reference is made to the following publication for consideration in the selection of a suitable material: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

Referring to the embodiment as depicted in FIG. 10, the elongated medical assembly 102 is configured to be inserted into a confined space defined by a living body (of the patient). The elongated medical assembly 102 is (preferably) impermeable by a bodily fluid located in the confined space defined by the living body.

Referring to the embodiment as depicted in FIG. 10, the guidewire 200 may include a shape-memory material configured to be manipulated and/or deformed followed by a return to the original shape that the shape-memory material was set in (prior to manipulation). Shape-memory materials (SMMs) are known and not further described in detail. Shape-memory materials are configured to recover their original shape from a significant and seemingly plastic deformation in response to a particular stimulus being applied to the shape-memory material. This is known as the shape memory effect (SME). Superelasticity (in alloys) may be observed once the shape-memory material is deformed under the presence (application) of a stimulus force.

Referring to the embodiments as depicted in FIG. 10 and FIG. 11, the puncture assembly 106 may include (and is not limited to) a radio frequency puncture device, such as the BAYLIS (TRADEMARK) POWERWIRE (REGISTERED TRADEMARK) radio frequency guidewire manufactured by BAYLIS MEDICAL COMPANY (headquartered in Canada). In accordance with another embodiment, the puncture assembly 106 may include (and is not limited to) a distal tip section presenting a mechanical cutting portion configured to form a puncture hole by physically moving the mechanical cutting portion into a biological feature.

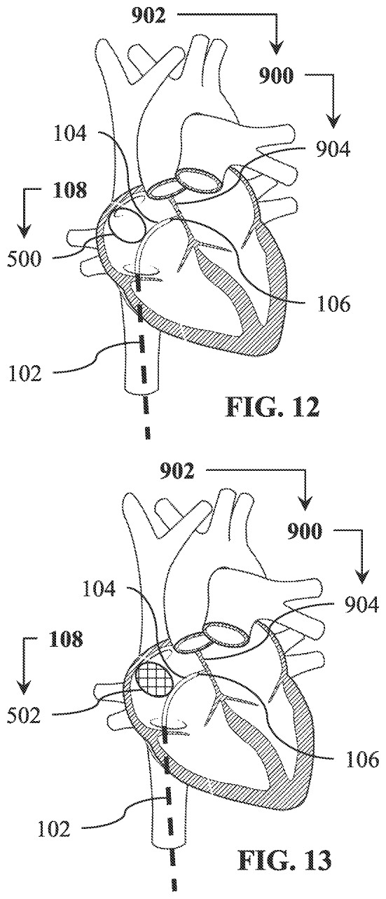

FIG. 12 and FIG. 13 depict side views of embodiments of the selectively expandable-and-contractible assembly 108 of FIG. 1.

Referring to the embodiments as depicted in FIG. 12 and FIG. 13, the selectively expandable-and-contractible assembly 108 includes a mechanically-expandable member 500, such as a frame structure (as depicted in FIG. 12), or a mesh structure 502 (as depicted in FIG. 13). The mechanically-expandable member 500 is, advantageously, hollow as to allow blood to pass without significant obstruction or turbulence to the hemodynamic flow. The mechanically-expandable member 500 may be in the form of a looped frame including at least one wire (or more than one wire) where at least one end of the wire(s) is (are) connected to the elongated medical assembly 102. The wire(s) may be made of nitinol or a shape-memory material that has been set to a shape (such as the shape of a curve, an oval or a circle, etc.). The shapes may have a range of radii and diameters suitable for its area and purpose of application (in terms of placement relative to the biological feature 900, etc.). For example, the mechanically-expandable member 500 may include a frame structure configured to expand in the inferior vena cava (IVC) and/or right atrium (RA) of the heart of the patient, and/or may have a different diameter and/or shape than a frame structure configured to expand in the right artery. The mechanically-expandable member 500 may include a looped frame including a wire with a shape set partial curve configured to be inserted into an inflation lumen at a proximal location of the elongated medical assembly 102, and the mechanically-expandable member 500 is configured to exit the elongated medical assembly 102 at a distal region thereof. The atraumatic tip portion (distal tip section) of the wire (that is, the elongated guidewire 200), once the mechanically-expandable member 500 has exited from the inflation lumen, may be advanced distally (in response to the movement of the mechanically-expandable member 500) such that the shape set curve (of the mechanically-expandable member 500) allows the atraumatic tip portion (of the elongated guidewire 200) to be (more easily) inserted into the biological feature 900 (such as the septum 904). The ports of the inflation lumens may be located about 90 degrees to about 270 degrees from each other. The wire (the elongated guidewire 200) is configured to be advanced from its proximal entrance until the tip of the wire (the elongated guidewire 200) exits at a proximal exit thereby forming a looped frame at the distal end. The ends of the wire (the elongated guidewire 200) may be advanced distally (or pushed) such that the looped frame is expanded to the surrounding anatomy (the biological feature 900). Another arrangement and/or method may include two wires (two instances of the elongated guidewire 200) each with an atraumatic magnetic tip portion, where each wire may be advanced through its respective inflation lumen from a proximal entrance to a distal exit. The magnetic attractive force between the magnetic tips may be configured to connect the two wires together. The joint may be retracted into an inflation lumen where it is securely held in place, and the two wires may be advanced distally as described above to expand the looped frame, etc. The mesh structure 502 (a mesh frame) may also be formed of a shape memory material set to recall its expanded shape. The mesh structure 502 may be mounted in its expanded state to the outer wall of the elongated medical assembly 102. A thin wall tube may be placed over the mesh structure 502 and the elongated medical assembly 102 to cause the mesh structure 502 to contract. After the elongated medical assembly 102 has been placed in the right artery (RA) of the heart, the thin wall tube may be retracted in the proximal direction to release the elongated medical assembly 102 to its expanded state.

The following is offered as further description of the embodiments, in which any one or more of any technical feature (described in the detailed description, the summary and the claims) may be combinable with any other one or more of any technical feature (described in the detailed description, the summary and the claims). It is understood hat each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the disclosure which does not materially modify the disclosure. Similarly, unless specifically made clear from its context, numerical values should be construed to include certain tolerances that the person skilled in the art would recognize as having negligible importance as they do not materially change the operability of the disclosure. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options may be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the disclosure. The foregoing has outlined the non-limiting embodiments (examples). The description is made for particular non-limiting embodiments (examples). It is understood that the non-limiting embodiments are merely illustrative as examples.

What is claimed is:

1. An apparatus, comprising:
an elongated medical assembly having a distal region; and
a puncture assembly being located proximate to the distal region; and
a selectively expandable-and-contractible assembly being located proximate to the distal region.

2. The apparatus of claim 1, wherein:
the selectively expandable-and-contractible assembly is configured to selectively adjust, at least in part, position of a distal region of the elongated medical assembly.

3. The apparatus of claim 1, wherein:
the selectively expandable-and-contractible assembly is configured to selectively stabilize, at least in part, a position of a distal region of the elongated medical assembly after the elongated medical assembly is positioned proximate to a biological feature.

4. The apparatus of claim 1, wherein:
the selectively expandable-and-contractible assembly includes a sac assembly configured to be deployable from the distal region in an uninflated condition to an inflated condition.

5. An apparatus, comprising:
an elongated medical assembly having:
a distal region configured to be positioned, at least in part, proximate to a biological feature of a patient; and
a proximal region spaced apart from the distal region, and the proximal region configured to be positioned outside of the patient after the distal region, in use, is positioned, at least in part, in the biological feature of the patient; and
a puncture assembly configured to be located proximate to the distal region, and the puncture assembly configured to form a puncture hole through the biological feature after the distal region, in use, is positioned, at least in part, proximate to the biological feature; and
a selectively expandable-and-contractible assembly configured to be deployable from the distal region in a contracted condition to an expanded condition; and
in the contracted condition, the selectively expandable-and-contractible assembly is contained within the distal region; and
in the expanded condition, the selectively expandable-and-contractible assembly is deployed, at least in part, from the distal region and proximate to the biological feature.

6. The apparatus of claim 5, wherein:
the selectively expandable-and-contractible assembly includes:
a sac assembly configured to be deployable from the distal region in an uninflated condition to an inflated condition; and
in the uninflated condition, the sac assembly is contained within the distal region; and
in the inflated condition, the sac assembly is deployed, at least in part, from the distal region and into the biological feature.

7. The apparatus of claim 5, wherein:
the elongated medical assembly defines an inflation lumen extending between the distal region and the proximal region; and
the inflation lumen is configured to be fluidly connectable, at the proximal region, to an inflation device positioned outside the patient.

8. The apparatus of claim 5, wherein:
the elongated medical assembly defines an inflation lumen extending between the distal region and the proximal region; and
the selectively expandable-and-contractible assembly includes a sac assembly configured to be expanded in response to injection of a fluid movable from an inflation device through the inflation lumen.

9. The apparatus of claim 8, wherein:
the inflation lumen is embedded in a wall of the elongated medical assembly.

10. The apparatus of claim 8, wherein:
the inflation device includes a syringe, an insufflator or a pump.

11. The apparatus of claim 5, wherein:
the selectively expandable-and-contractible assembly includes:
a sac assembly configured to impart an action force against the biological feature after the sac assembly is deployed so that the action force, in use, urges a reaction force to become imparted to the distal region, which causes the distal region to deflect, thereby changing a reach of the distal region.

12. The apparatus of claim 5, wherein:
the selectively expandable-and-contractible assembly includes:
a sac assembly configured to rest against the biological feature to stabilize the elongated medical assembly during deployment of the puncture assembly.

13. The apparatus of claim 5, wherein:
the selectively expandable-and-contractible assembly includes:
a sac assembly configured to be embedded on one side of a circumference of the elongated medical assembly.

14. The apparatus of claim 5, wherein:
the selectively expandable-and-contractible assembly includes:
a sac assembly configured to be embedded around an entire circumference of the elongated medical assembly.

15. The apparatus of claim 5, wherein:
the elongated medical assembly defines an inflation lumen extending between the distal region and the proximal region; and
the inflation lumen is configured to be fluidly connectable, at the proximal region, to an inflation device positioned outside the patient, in which the inflation device is configured to provide a contrast fluid to the inflation lumen.

16. The apparatus of claim 5, wherein:
the selectively expandable-and-contractible assembly includes a sac assembly configured to be visible under fluoroscopy when filled with a contrast fluid.

17. The apparatus of claim 5, wherein:
the elongated medical assembly includes:
an elongated sheath assembly; an elongated dilator assembly configured to be received within sheath assembly; and
an elongated guidewire configured to be received within the elongated dilator assembly.

18. The apparatus of claim 5, wherein:
the selectively expandable-and-contractible assembly is configured to deflect, at least in part, the distal region of the elongated medical assembly or provide, at least in part, additional stability of the elongated medical assembly.

19. The apparatus of claim 5, wherein:
the selectively expandable-and-contractible assembly includes a mechanically-expandable member or a mesh structure.

20. A method of using an elongated medical assembly having a distal region configured to be positioned, at least in part, proximate to a biological feature of a patient, and a puncture assembly being located proximate to the distal region, and a selectively expandable-and-contractible assembly being located proximate to the distal region, the method comprising:
deploying the selectively expandable-and-contractible assembly at the distal region in a storage condition to a deployment condition;
in the storage condition, the selectively expandable-and-contractible assembly is contained within the distal region; and
in the deployment condition, the selectively expandable-and-contractible assembly is deployed, at least in part, from the distal region and is positioned proximate to the biological feature
